(19) United States
(12) Patent Application Publication
(10) Pub. No.: US 2009/0214682 A1
(43) Pub. Date: Aug. 27, 2009

(54) Composition and methods for weight loss in a subject

(76) Inventors: Marvin A. Heuer, (US); Ken Clement, (US)

Correspondence Address: IOVATE HEALTH SCIENCE RESEARCH INC., 381 North Service Road West, Oakville, ON, L6M 0H4, CA

(21) Appl. No.: 12/316,648

(22) Filed: Dec. 15, 2008

Related U.S. Application Data
(60) Provisional application No. 61/030,712, filed on Feb. 22, 2008.

(51) Int. Cl. A61K 31/337; A61K 31/522; A61K 31/555; A61K 36/605; A61K 31/353; A61K 31/437; A61P 3/04

(52) U.S. Cl. 424/769; 514/449; 514/263.34; 514/188; 514/456; 514/280

(57) ABSTRACT

The present invention relates generally to weight loss compositions and more particularly to orally administrable weight loss compositions for supporting weight loss in a subject in need thereof. Specifically, the present invention relates to compositions and methods comprising various combinations of orlistat and substances selected from methylxanthines, extracts of Morus sp., chromium polynicotinate, hydroxycitric acid or derivatives of hydroxycitric acid, flavanols, and yohimbine or derivatives of yohimbine.

### RELATED APPLICATIONS

The present application is related to and claims benefit of priority to U.S. Provisional Application No. 61/030,712 entitled “Compositions and Methods for Weight Loss in a Mammal” filed Feb. 22, 2008, the disclosure of which is hereby fully incorporated by reference.

### FIELD OF THE INVENTION

The present invention relates generally to oral compositions, and more particularly to oral compositions for supporting weight loss in a subject in need thereof. Specifically, the present invention relates to compositions and methods comprising various combinations of orlistat and substances selected from methylxanthines, extracts of Morus sp., chromium polynicotinate, hydroxycitric acid or derivatives of hydroxycitric acid, flavanols, and yohimbine or derivatives of yohimbine.

### BACKGROUND OF THE INVENTION

Obesity has become an increasingly widespread and predominant health concern. According to the World Health Organization (WHO) obesity is considered a multifactorial chronic disease which is increasing in frequency. Obesity, a condition of excessive body fat, generally results from more energy (food) being consumed than is being used. Stemming from excessive body fat, several health-related concerns such as increased morbidity have been linked to obesity and being overweight as well as hypertension, coronary heart disease, type 2 diabetes mellitus, stroke and even some forms of cancer.

Not surprisingly, a great deal of effort has gone into addressing the problems created by excessive weight. Fad diets, diet pills, diet foods, liposuction, intestinal bypass surgery, and lifestyle changes have all been advanced as possible solutions to the problem of weight loss in overweight individuals, some with greater success than others.

Given the prevalence and serious problems associated with obesity, and the significant drawbacks associated with intrusive surgical procedures and the lack of success of fad diets, a need exists for a high-compliance weight loss composition that is easy to administer to persons that are overweight or obese to reduce weight gain, cause weight loss, and reduce body mass index to acceptably healthy levels.

### SUMMARY OF THE INVENTION

The present invention is directed towards a weight loss composition comprising an effective amount of orlistat and an effective amount of a methylxanthine. Both a composition and a method are provided by the present disclosure.

In an additional embodiment of the present invention the weight loss composition comprises an effective amount of orlistat and an effective amount of an extract of Morus sp. Both a composition and a method are provided by the present disclosure.

In a further embodiment of the present invention the weight loss composition comprises an effective amount of orlistat and an effective amount of chromium polynicotinate. Both a composition and a method are provided by the present disclosure.

In still a further embodiment of the present invention the weight loss composition comprises an effective amount of orlistat and an effective amount of hydroxycitric acid or derivatives of hydroxycitric acid. Both a composition and a method are provided by the present disclosure.

In yet a further embodiment of the present invention the weight loss composition comprises an effective amount of orlistat and a flavanol and may further comprise yohimbine or derivatives of yohimbine. Both compositions and methods are provided by the present disclosure.

### DETAILED DESCRIPTION OF THE INVENTION

In the following description, for the purposes of explanations, numerous specific details are set forth in order to provide a thorough understanding of the present invention. It will be apparent, however, to one of ordinary skill in the art that the present invention may be practiced without these specific details.

The present invention is directed towards a weight loss composition and method for supporting weight loss in a subject by acting to substantially simultaneously decrease the absorption of ingested fats by inhibiting pancreatic lipases, and increase thermogenesis and energy expenditure via increased levels of CAMP.

An additional embodiment of the present invention is directed towards a weight loss composition and method for supporting weight loss in a subject by acting to concomitantly decrease the absorption of ingested fats by inhibiting pancreatic lipases, and decrease the absorption of ingested carbohydrates by inhibiting the activity of α-glucosidases.

A further embodiment of the present invention is directed towards a weight loss composition and method for supporting weight loss in a subject by acting to concomitantly decrease the absorption of ingested fats by inhibiting pancreatic lipases, and lowering serum glucose and free fatty acid levels by facilitating increased glucose tolerance factor levels.

An additional embodiment of the present invention is directed towards a weight loss composition and method for supporting weight loss in a subject by acting to substantially simultaneously decrease the absorption of ingested fats by inhibiting pancreatic lipases, curb or control appetite, and inhibit fatty acid synthesis.

A further embodiment of the present invention is directed towards a weight loss composition and method for supporting weight loss in a subject by acting to substantially simultaneously decrease the absorption of ingested fats by inhibiting pancreatic lipases, reduce fat digestion and increase energy expenditure. Additionally this embodiment of the present invention may contain an additional ingredient which acts to reduce food intake and increase energy expenditure, via increased leptin levels.

Extracts suitable for use in the present invention may be produced by extraction methods as are known and accepted in the art such as alcoholic extraction, aqueous extractions, carbon dioxide extractions, for example.

As used herein, ‘orlistat’ refers to the chemical N-formyl-L-leucine-1-[[(2S,3S)-3-hexyl-4oxo-2-oxetanyl]methyl]dodecyl ester, (CAS Registry No. 96829-58-2), also known as N-formyl-L-leucine ester with (3S,4S)-3-hexyl-4-[(2S)-2-hydroxy-tridecyl]-2-oxetanone, tetrahydrolipstatin, or orlipastat.

As used herein, ‘caffeine’ refers to the chemical 3,7-dihydro-1,3,7-trimethyl-1H-purine-2,6-dione, (CAS Registry No. 58-08-2), also known as 1,3,7-trimethylxanthine, 1,3,7-trimethyl-2,6-dioxopurine, caffeine, theine, guaranine, mateine, or methyltheobromine. Additionally, as used herein, ‘caffeine’ also includes derivatives of caffeine such as esters, amides, and salts, as well as other derivatives, including derivatives having substantially similar pharmacoproperties to caffeine upon metabolism to an active form.

As used herein, ‘theophylline’ refers to the chemical 3,7-dihydro-1,3-dimethyl-1H-purine-2,6-dione, (CAS Registry No. 58-55-9), also known as 1,3-dimethylxanthine, accurbron, aerobin, aerolate, afonilum, armophylline, or theostat. Additionally, as used herein, ‘theophylline’ also includes derivatives of theophylline such as esters, amides, and salts, as well as other derivatives, including derivatives having substantially similar pharmacoproperties to theophylline upon metabolism to an active form.

As used herein, ‘theobromine’ refers to the chemical 3,7-dihydro-3,7-dimethyl-1H-purine-2,6-dione, (CAS Registry No. 83-67-0), also known as 3,7-dimethylxanthine or xantheose. Additionally, as used herein, ‘theobromine’ also includes derivatives of theobromine such as esters, amides, and salts, as well as other derivatives, including derivatives having substantially similar pharmacoproperties to theobromine upon metabolism to an active form.

As used herein, ‘hydroxycitric acid’ refers to the chemical 1,2-dihydroxypropane-1,2,3-tricarboxylic acid, (CAS Registry No. 6205-14-7), also known as hydroxycitrate. Additionally, as used herein, ‘hydroxycitric acid’ also includes derivatives of hydroxycitric acid such as esters, amides, and salts, as well as other derivatives, including derivatives having substantially similar pharmacoproperties to hydroxycitric acid upon metabolism to an active form.

As used herein, the term ‘flavanol’ refers primarily to catechin, epicatechin, and their derivatives. These derivatives include the sugar salts, sugar esters, gallate esters, and other edible physiologically available derivatives. The preferred flavanols are catechin, gallocatechin, catechingallate, gallocatechingallate, epicatechin, epigallocatechin, epicatechingallate, and epigallocatechingallate.

As used herein, ‘yohimbine’ refers to the chemical (16a,17a)-17-hydroxy-yohimban-16-carboxylic acid methyl ester, (CAS Registry No. 14648-5), also known as quebrachine, corynine or aphrodine. Additionally, as used herein ‘yohimbine’ also includes derivatives of yohimbine such as esters, amides, and salts, as well as other derivatives, including derivatives having substantially similar pharmacoproperties to yohimbine upon metabolism to an active form.

As used herein, the term ‘subject’ refers to mammals and non-mammals. Mammals refers to any member of the mammalia class including, but not limited to, humans; non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. Preferably, the subject is a human.

As used herein, the term ‘composition’ includes dietary supplements, diet supplements, nutritional supplements, supplemental compositions and supplemental dietary compositions or those similarly envisioned and termed compositions not belonging to the conventional definition of pharmaceutical interventions as is known in the art. Furthermore, ‘compositions’ as disclosed herein belong to category of compositions having at least one physiological function when administered to a subject by conventional routes of administration.

Alternatively, formulations and compositions belonging to the present invention may be considered to be nutraceuticals. As used herein, the term ‘nutraceutical’ is recognized and used in the art to describe a specific chemical compound or combination of compounds found in, organic matter for example, which may prevent, ameliorate or otherwise confer benefits against an undesirable condition. As is known in the art, the term ‘nutraceutical’ is used to refer any substance that is a food, a part of food, or an extract of food which is suitable for consumption by an individual and providing physiological benefit which may be medical or health-related. Furthermore, the term has been used to refer to a product isolated, extracted or purified from foods or naturally-derived material suitable for consumption by an individual and usually sold in medicinal forms, such as caplets, tablet, capsules, soft-gel™ caplets, gel-caps and the like, not associated with food.

Orlistat

Tetrahydrolipstatin, or 1-(3-hexyl-4-oxo-oxetan-2-yl)tridecan-2-yl, 2-formyl-amino-4-methyl-pentanoate, more commonly referred to as orlistat, is a natural derivative of lipstatin, which is a natural inhibitor of pancreatic lipases. Due to the simplicity and the stability of orlistat, it was developed into a drug for treating obesity as opposed to lipstatin.

In order for the mammalian body to absorb ingested fats they must first be broken down by pancreatic lipases in the gastrointestinal tract. These lipases convert triglycerides in foods to monoglycerides and free fatty acids, which are readily absorbable forms of fat. Orlistat binds to and inhibits the actions of pancreatic lipases, thereby reducing the amount of ingested fats that are absorbed. Since undigested fat cannot be absorbed by the body, it passes naturally out of the body. Fats are the most calorie dense nutrients when compared to carbohydrates and proteins, so reducing the absorption of fats will have the most significant impact on total caloric intake.

Administration of orlistat to individuals in which weight loss is notoriously difficult, i.e. persons with type 2 diabetes, lead to more significant weight loss then the placebo group. Additionally, treatment with orlistat over a one year period showed greater improvements then placebo in LDL cholesterol levels and total serum cholesterol.

Orlistat was approved by the FDA, for over-the-counter (OTC) administration in doses of 60 mg, in August 2007. The lipase inhibitor inhibits the absorption of about 25% of ingested fats and shows an average weight loss of about 5% of the initial body weight over a period of 16 weeks. The OTC dose has about 85% the efficacy of the prescription dose of 120 mg for weight loss. The lower dosage of 60 mg is generally better tolerated then the larger dose and the gastrointestinal side effects are minimal when less than 30% of an individual's energy is from fat.

Although the present invention is not to be limited by any theoretical explanation, it is herein understood by the inventors that inclusion of orlistat in a weight loss composition will support weight loss in a subject by inhibiting the uptake of ingested fats from the gastrointestinal tract via inhibition of pancreatic lipases. Inhibition of lipases in the gastrointestinal tract causes a decline in fat degradation, which is necessary for absorption, thus leading to decreased absorption of fats. Decreased absorption of dietary fats will help to reduce caloric intake, thereby acting to support weight loss in a subject.

In an embodiment of the present invention which is set forth in greater detail in the examples below, the composition comprises an effective amount of orlistat. A serving of the composition comprises from about 10 mg to about 60 mg of orlistat. In the preferred dosage of the present invention, a serving of the composition comprises about 60 mg of orlistat.

Methylxanthines

Methylxanthines are methylated forms of the purine base, xanthine, which is found throughout most tissues and fluids of various organisms. Common methylxanthine derivatives include caffeine, paraxanthine, theophylline, and theobromine. Methylxanthine derivatives are nonselective adenosine receptor antagonists, central nervous system stimulants, and are often used to ward off drowsiness and restore alertness.

Administration of the methylxanthine derivatives, such as caffeine, theophylline and theobromine, to rats fed a high fat diet, showed that over a period of 12 weeks the body fat of the rats, which were fed methylxanthines, was generally reduced. The methylxanthine-fed groups, particularly the caffeine-fed group, showed a significant decrease in both abdominal adipose tissue weight and serum triglyceride levels as compared to the control group.

Methylxanthine derivatives, most commonly caffeine, are widely used to control weight through multiple mechanisms. Significant weight loss related to caffeine supplementation has been observed in obese women, which may be, at least in part, due to increased lipolysis as fat is metabolized. Methylxanthines have additionally been shown to increase the basal metabolic rate in humans wherein this also adds to its weight-lowering effects via increased energy expenditure.

Methylxanthines can increase levels of intracellular cAMP, which are important signaling molecules involved in many metabolic processes including thermogenesis, via antagonism of adenosine receptors. Additionally, methylxanthines, particularly caffeine, also facilitate increased levels of CAMP by inhibiting phosphodiesterases responsible for the degradation of CAMP. The resultant preservation of CAMP, via at least the two mechanisms discussed above, result in increased release of epinephrine and norepinephrine. Increases in levels of epinephrine and norepinephrine will yield enhanced adrenergic signaling thereby inducing lipolysis, where thermogenesis can expend energy leading to body fat loss.

Although the present invention is not to be limited by any theoretical explanation, it is herein understood by the inventors that inclusion of methylxanthines in a weight loss composition will support weight loss in a subject by increasing energy expenditure through thermogenesis. Antagonism of adenosine receptors and phosphodiesterase inhibition, by methylxanthines, results in increased levels of CAMP thereby leading to increases in thermogenesis and thus energy expenditure.

In an embodiment of the present invention which is set forth in greater detail in the examples below, the composition comprises an effective amount of caffeine. A serving of the composition comprises from about 1 mg to about 400 mg of caffeine. In the preferred dosage of the present invention, a serving of the composition comprises about 200 mg of caffeine, preferably caffeine anhydrous.

In an additional embodiment of the present invention which is set forth in greater detail in the examples below, the composition comprises an effective amount of theobromine. A serving of the composition comprises from about 5 mg to about 350 mg of theobromine. In the preferred dosage of the present invention, a serving of the composition comprises about 75 mg of theobromine, the theobromine most preferably being from an extract of Theobroma cacao.

Extract of Morus sp.

The genus Morus refers to an edible plant, commonly known as mulberry, which is rich in flavonoids with antioxidant activity and has long been a staple of Chinese medicine. There are over 150 species names that have been published for Mulberry. However, the following are the species that are generally accepted: alba, australis, celtidifolia, insignis, mesozygia, microphylla, nigra, and rubra.

Oral administration of mulberry, preferably Morus alba, leaves have been shown to result in weight loss and reduced increases in glucose levels after eating, which has been linked to reduced carbohydrate absorption. Furthermore, carbohydrate absorption has been shown to be attenuated, in healthy humans, by tea extracts comprising mulberry.

The malabsorption of carbohydrates has been attributed to specific constituents of mulberry tea extract that inhibit α-glucosidase activity. The aforementioned study used the breath hydrogen concentration technique to measure carbohydrate absorption in individuals consuming a carbohydrate meal either with or without a tea extract beverage of mulberry. Breath hydrogen concentration was shown to be significantly higher in the individuals that consumed the mulberry tea extract beverage with the carbohydrate meal, thus indicating an inhibition in the absorption of carbohydrates. It is likely that inhibition of glucosidases in the gastrointestinal tract causes a decline in carbohydrate degradation, which is necessary for absorption, thus leading to decreased absorption of carbohydrates.

Co-ingestion of mulberry tea leaf extract and 75 g of sucrose has been shown to significantly reduce increase of blood glucose levels as compared those receiving a placebo and 75 g of sucrose. Additionally, reduction in blood glucose levels below that of fasting seen in the control group at the tail of the study period was not observed in the treatment group. It has been suggested that inhibition of intestinal sucrase activity causes the reduction in blood glucose levels that is observed after co-ingestion of mulberry tea leaf extract and 75 g of sucrose.

Although the present invention is not to be limited by any theoretical explanation, it is herein understood by the inventors that inclusion of extracts of Morus, particularly extracts of Morus alba, in a weight loss composition will support weight loss in a subject by inhibiting the uptake of ingested carbohydrates from the gastrointestinal tract by inhibition of gluosidases. Inhibition of glucosidases in the gastrointestinal tract causes a decline in carbohydrate degradation necessary for absorption, thus leading to decreased absorption of carbohydrates.

In an embodiment of the present invention which is set forth in greater detail in the examples below, the composition comprises an effective amount of an extract of Morus sp., preferably an extract of Morus alba leaves. A serving of the composition comprises from about 0.1 g to about 2.0 g of an extract of Morus sp. In the preferred dosage of the present invention, a serving of the composition comprises about 1.2 g of an extract of Morus sp., preferably an extract of Morus alba leaves.

Chromium Polynicotinate

Chromium is an essential trace mineral that is used to control blood sugar levels by aiding insulin, which can help control or reduce weight. Chromium, as it is poorly absorbed by the body, must be combined with a more efficiently absorbed compound such as niacin, typically multiple niacin molecules, i.e. polynicotinate. Chromium likely exerts its main function as a component of the glucose tolerance factor, which is involved in insulin sensitivity. Increased levels of glucose tolerance factor have been linked to decreased serum glucose levels and free fatty acids in blood.

Chromium has been shown clinically to increase lean mass and reduce body fat when combined with exercise. Furthermore, chromium has also been shown to increase HDL (‘good’) cholesterol.

Moreover, in an animal study designed to assess the effectiveness of chromium polynicotinate supplementation on Metabolic Syndrome, as defined by a cluster of risk factors including obesity, increased abdominal fat, insulin resistance, hypertension and blood lipid abnormalities, it was shown that compared to control groups chromium polynicotinate was effective in lowering systolic blood pressure. This study also observed less free radical damage in the liver and kidney tissue as compared to controls, indicating antioxidant properties. Further to the animal studies, a clinical study has shown that niacin-bound chromium supplementation for 8 weeks is able to result in a significant loss of body fat in overweight subjects as compared to a placebo group while sparing muscle from loss.

Although the present invention is not to be limited by any theoretical explanation, it is herein understood by the inventors that inclusion of chromium polynicotinate in a weight loss composition will support weight loss in a subject by facilitating an increase in glucose tolerance factor levels. Increased levels of glucose tolerance factor act to reduce levels of glucose and free fatty acids in the serum.

In an embodiment of the present invention which is set forth in greater detail in the examples below, the composition comprises an effective amount of chromium polynicotinate. A serving of the composition comprises from about 0.1 mg to about 5 mg of chromium polynictotinate. In the preferred dosage of the present invention, a serving of the composition comprises about 1.33 mg of chromium polynicotinate.

Hydroxycitrc Acid

Hydroxycitric acid (HCA) is extracted from the fruits of the Garcinia Cambogia, Garcinia indica, and Garcinia atrovirdis plants. It is used to control weight by virtue of its ability to inhibit fat production and suppress appetite.

U.S. patent application Ser. No. 10/425,428, entitled “Hydroxycitric acid salt composition and method of making” describes a method for producing stable and pure calcium and potassium hydroxycitric acid salts which are essentially tasteless. The hydroxycitric acid salts are prepared by a process that includes treating an aqueous extract of Garcinia cambogia with a liquid quaternizing agent. The extract is further treated with potassium hydroxide or sodium hydroxide. Potassium hydroxycitrate can then be recovered and the sodium solution is further treated with a calcium salt to yield a recoverable calcium hydroxycitrate.

HCA has been shown to inhibit fatty acid synthesis and repress appetite in rats. It is additionally known to be a competitive inhibitor of ATP:citrate lyase; an enzyme necessary for the conversion of carbohydrates into fat. By inhibiting this enzyme, HCA blocks the body's ability to produce fat from carbohydrate sources.

Several human clinical trials have demonstrated the safety and beneficial effects of HCA in terms of weight management. HCA clinical studies have shown that its administration is able to produce a reduction in caloric intake, while further increasing fat oxidation during exercise in untrained men and women. A randomized controlled trial which combined data from two earlier trials demonstrated that daily HCA supplementation with moderate exercise over a period of 8 weeks resulted in increased weight loss as compared to a placebo group and an improved blood cholesterol profile. Additionally, serotonin levels were significantly increased by HCA, thereby resulting in decreased hunger signals, particularly signals associated with carbohydrate cravings. This could result in a reduction of carbohydrate cravings.

Although the present invention is not to be limited by any theoretical explanation, it is herein understood by the inventors that inclusion of hydroxycitric acid or derivatives of hydroxycitric acid in a weight loss composition will support weight loss in a subject by acting to curb or control appetite and inhibit fatty acid synthesis. By inhibiting the enzyme responsible for converting excess carbohydrates into fat, HCA acts to reduce body fat accumulation resulting from ingestion of excess calories. Additionally, HCA acts to increase serotonin levels leading to reduction of hunger signals, particularly those associated with carbohydrate cravings.

In an embodiment of the present invention which is set forth in greater detail in the examples below, the composition comprises an effective amount of hydroxycitric acid. A serving of the composition comprises from about 0.50 g to about 3.0 g of hydroxycitric acid. In the preferred dosage of the present invention, a serving of the composition comprises about 0.933 g of hydroxycitric acid, particularly from an extract of Garcina cambogia.

Flavanols

Flavanols are a subgroup of a class of plant secondary metabolites often collectively referred to as flavonoids or bioflavonoids. They are found widely throughout nature, most often concentrated in fruits, vegetables, wines, teas and cocoa. Flavonoids have been shown to have anti-allergic, anti-inflammatory and anti-cancer activity. Flavonoids are present in a large number of plants for various purposes ranging from pigmentation to protection from attack by microbes and insects.

Substances, such as green tea, which are known to contain large amounts of various flavanols, have been shown to be effective in aiding weight loss. This effect may be due to two activities. Green tea both reduces fat digestion and increases energy expenditure. Fat stores may provide the energy necessary for the increase in energy expenditure via the oxidation of fat, consequently leading to thermogenesis. The thermogenic activity of green tea may additionally be greatly enhanced by synergistic cooperation with additionally added caffeine. In the regard of the p-oxidation of fats owing to green tea is a result of activities at adenosine receptors and their effect on an increase in cyclic adenosine monophosphate (cAMP).

Additionally, the mechanism of action of green tea may also be, due to an increase in norepinephrine. Flavanols, i.e. catechins, found in green tea, are known to inhibit catechol-O-methyl-transferase (COMT), an enzyme which degrades norepinephrine. In turn, norepinephrine inhibits the degradation as well as increases the production of cyclic adenosine monophosphate (cAMP). Furthermore, increasing norepinephrine levels by the inhibition of norepinephrine uptake results in increased weight loss in both lean and obese mice as evidenced in animal studies. Stemming from increased norepinephrine levels is the result of an increased presence of CAMP which leads to greater activation of protein kinase A, which subsequently activates lipases found in adipose tissue.

Although the present invention is not to be limited by any theoretical explanation, it is herein understood by the inventors that inclusion of flavanols in a weight loss composition will support weight loss in a subject by concomitantly reducing fat digestion and increasing energy expenditure. Flavanols inhibit COMT, which acts to degrade norepinephrine, thereby increasing levels of norepinephrine. Norepinephrine inhibits the degradation and enhances production of cAMP.

In an embodiment of the present invention which is set forth in greater detail in the examples below, the composition comprises an effective amount of a flavanol. A serving of the composition comprises from about 100 mg to about 700 mg of a flavanol. In the preferred dosage of the present invention, a serving of the composition comprises about 200 mg of a flavanol.

Yohimbine

Yohimbine is a naturally occurring alkaloid derived from the bark of the African tree, Pausinysatlia yohimbe. Traditionally, yohimbine has been used as an aphrodisiac in traditional medicine and additionally as a treatment for erectile dysfunction. Yohimbine is a competitive α₂-adrenerigic receptor antagonist. These receptors mediate a variety of functions in the body including food intake,

Yohimbine has been shown to significantly increase leptin and glucose levels, when administered to rats, compared to placebo. Additionally, it was noted that the body weight of yohimbine treated rats was significantly reduced as compared to untreated rats. Leptin acts as a signal from adipose stores to the hypothalamus to decrease food intake and increase energy expenditure. Therefore increased levels of leptin in the blood will act to decrease the intake of food while concomitantly causing the body to increase energy expenditure leading to loss of body weight.

Although the present invention is not to be limited by any theoretical explanation, it is herein understood by the inventors that inclusion of yohimbine or derivatives of yohimbine in a weight loss composition will support weight loss in a subject by concomitantly reducing food intake and increasing energy expenditure. By increasing leptin levels in the body, yohimbine acts to reduce body fat accumulation resulting from ingestion of excess calories. Additionally, increased leptin levels will increase energy expenditure, leading to loss of body weight.

In an embodiment of the present invention which is set forth in greater detail in the examples below, the composition comprises an effective amount of yohimbine or derivative of yohimbine. A serving of the composition comprises from about 1 mg to about 20 mg of yohimbine or derivative of yohimbine. In the preferred dosage of the present invention, a serving of the composition comprises about 5.5 mg of yohimbine or derivative of yohimbine.

Although the present invention is not to be limited by any theoretical explanation, it is herein understood by the inventors that a weight loss composition comprising orlistat and methylxanthines will support weight loss in a subject by acting to substantially simultaneously decrease the absorption of ingested fats by inhibiting pancreatic lipases, and increase thermogenesis and energy expenditure via increased levels of cAMP.

Additionally, although the present invention is not to be limited by any theoretical explanation, it is herein understood by the inventors that a weight loss composition comprising orlistat and an extract of Morus sp. will support weight loss in a subject by acting to concomitantly decrease the absorption of ingested fats by inhibiting pancreatic lipases, and decrease the absorption of ingested carbohydrates by inhibiting the activity of α-glucosidases.

Furthermore, although the present invention is not to be limited by any theoretical explanation, it is herein understood by the inventors that a weight loss composition comprising orlistat and chromium polynicotinate will support weight loss in a subject by acting to concomitantly decrease the absorption of ingested fats by inhibiting pancreatic lipases, and lower serum glucose and free fatty acid levels by facilitating increased glucose tolerance factor levels.

Additionally, although the present invention is not to be limited by any theoretical explanation, it is herein understood by the inventors that a weight loss composition comprising orlistat and hydroxycitric acid or derivative of hydroxycitric acid will support weight loss in a subject by acting to substantially simultaneously decrease the absorption of ingested fats by inhibiting pancreatic lipases, curb or control appetite, and inhibit fatty acid synthesis.

Furthermore, although the present invention is not to be limited by any theoretical explanation, it is herein understood by the inventors that a weight loss composition comprising orlistat and flavanols will support weight loss in a subject by acting to substantially simultaneously decrease the absorption of ingested fats by inhibiting pancreatic lipases, reduce fat digestion and increase energy expenditure by increasing levels of norepinephrine and cAMP in the body. Additionally, inclusion of yohimbine in the weight loss composition will support weight loss by reducing food intake and increasing energy expenditure via increased leptin levels.

According to various embodiments of the present invention, the weight loss composition may be consumed in any form. For instance, the dosage form of the weight loss composition may be provided as, e.g., a capsule, a liquid capsule, a tablet, a caplet, gel-cap, or soft-gel. The preferred dosage forms of the present invention are as a liquid capsule.

In addition to the foregoing, weight loss compositions of the present invention include formulations further comprising additional active ingredients and/or inactive ingredients, including solvents, diluents, suspension aids, thickening or emulsifying agents, sweeteners, flavorings, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and which may also be suitable for use in formulations of the present invention. Except insofar as any conventional carrier medium is incompatible with the ingredients of the invention, such as by producing any undesirable effect or otherwise interacting in a deleterious manner with any other ingredient(s) of the formulation, its use is contemplated to be within the scope of this invention.

Although the following example illustrates the practice of the present invention in one of its embodiments, the example should not be construed as limiting the scope of the invention. Other embodiments will be apparent to one of skill in the art from consideration of the specifications and example.

### EXAMPLES

### Example 1

The weight loss composition can be provided in three servings per day as one gel-cap per serving. In an embodiment of the present invention, each serving of the weight loss composition can comprise:

about 60 mg of orlistat, and about 200 mg of caffeine anhydrous.

### Example 2

The weight loss composition can be provided in two servings per day as one soft-gel per serving. In an embodiment of the present invention, each serving of the weight loss composition can comprise:

about 60 mg of orlistat, and about 350 mg of caffeine anhydrous.

### Example 3

The weight loss composition can be provided in three servings per day as two caplets per serving. In an embodiment of the present invention, each serving of the weight loss composition can comprise:

about 40 mg of orlistat, and about 75 mg of theobromine.

### Example 4

The weight loss composition can be provided in two servings per day as two tablets per serving. In an embodiment of the present invention, each serving of the weight loss composition can comprise:

about 60 mg of orlistat, and about 1.255 g of Theobroma cacao extract standardized to 6% theobromine.

### Example 5

The weight loss composition can be provided in three servings per day as two soft-gel per serving. In an embodiment of the present invention, each serving of the weight loss composition can comprise:

about 60 mg of orlistat, and about 1.2 g of Morus alba extract.

### Example 6

The weight loss composition can be provided in two servings per day as one gel-cap per serving. In an embodiment of the present invention, each serving of the weight loss composition can comprise:

about 60 mg of orlistat, and about 1.0 g of Morus alba extract.

### Example 7

The weight loss composition can be provided in three servings per day as one tablet per serving. In an embodiment of the present invention, each serving of the weight loss composition can comprise:

about 60 mg of orlistat, and about 1.33 mg of chromium polynicotinate.

### Example 8

The weight loss composition can be provided in two servings per day as one liquid capsule per serving. In an embodiment of the present invention, each serving of the weight loss composition can comprise:

about 60 mg of orlistat, and about 2.75 mg of chromium polynicotinate.

### Example 9

The weight loss composition can be provided in three servings per day as two gel-caps per serving. In an embodiment of the present invention, each serving of the weight loss composition can comprise:

about 60 mg of orlistat, and about 1.5556 g of Garcinia cambogia extract standardized to 60% hydroxycitric acid.

### Example 10

The weight loss composition can be provided in two servings per day as two tablets per serving. In an embodiment of the present invention, each serving of the weight loss composition can comprise:

about 60 mg of orlistat, and about 500 mg of calcium hydroxycitric acid.

### Example 11

The weight loss composition can be provided in two servings per day as two caplets per serving. In an embodiment of the present invention, each serving of the weight loss composition can comprise:

about 60 mg of orlistat, and about 200 mg of epigallocatechingallate.

### Example 12

The weight loss composition can be provided in two servings per day as two tablet per serving. In an embodiment of the present invention, each serving of the weight loss composition can comprise:

about 60 mg of orlistat, about 225 mg of epigallocatechingallate, and about 225 mg of catechin.

Extensions and Alternatives

In the foregoing specification, the invention has been described with specific embodiments thereof; however, it will be evident that various modifications and changes may be made thereto without departing from the broader spirit and scope of the invention.

All publications which are cited herein are hereby specifically incorporated by reference into the disclosure for the teachings for which they are cited.

What is claimed:

1. A weight loss composition comprising:
orlistat and one or more methylxanthine.

2. The composition of claim 1, wherein the one or more methylxanthine is selected from the group consisting of caffeine, theophylline, and theobromine.

3. A method for supporting weight loss in a subject comprising providing to said subject the composition of claim 1.

4. The method of claim 3, wherein the one or more methylxanthine is caffeine anhydrous.

5. The method of claim 3, wherein the theobromine is from an extract of Theobroma cacao.

6. A weight loss composition comprising:
orlistat and an extract of Morus sp.

7. The composition of claim 6, wherein the extract is Morus alba.

8. The composition of 7, wherein the amount of orlistat is about 60 mg and the amount of the extract of Morus alba is about 1.2 g.

9. A method for supporting weight loss in a subject comprising providing to said subject the composition of claim 6.

10. A weight loss composition comprising: orlistat and chromium polynicotinate.

11. The composition of claim 10, wherein the amount of orlistat is about 60 mg and the amount of chromium polynicotinate is about 1.33 mg.

12. A method for supporting weight loss in subject comprising providing to said subject the composition of claim 10.

13. A weight loss composition comprising:
orlistat and hydroxycitric acid.

14. The composition of claim 13, wherein the source of hydroxycitric acid is extracted from the group consisting of an extract of Garcinia cambogia, an extract of Garcinia indica, and an extract of Garcinia atrovirdis.

15. The composition of claim 13, wherein the hydroxycitric acid is a metal salt of hydroxycitric acid.

16. The composition of claim 15, wherein the metal is selected from the group consisting of zinc, magnesium, sodium, potassium, and calcium.

17. The composition of claim 13, wherein the amount of orlistat is about 60 mg and the amount of the hydroxycitric acid is about 933 mg.

18. A method for supporting weight loss in a subject comprising providing to said subject the composition of claim 13.

19. The method of claim 18, wherein the hydroxycitric acid is extracted from the group consisting of an extract of Garcinia cambogia, an extract of Garcinia indica, and an extract of Garcinia atrovirdis.

20. The method of claim 18, wherein the hydroxycitric acid is a metal salt of hydroxycitric acid.

21. A weight loss composition comprising orlistat and a flavanol.

22. The composition of claim 21, wherein the flavanol is selected from the group consisting of catechin, gallocatechin, catechingallate, gallocatechingallate, epicatechin, epigallocatechin, epicatechingallate, epigallocatechingallate and combinations thereof.

23. The composition of claim 21 further comprising yohimbine.

24. The composition of claim 21, wherein the amount of orlistat per day is about 180 mg and the amount of flavanol per day is from about 350 mg to about 700 mg.

25. The composition of claim 23, wherein the amount of yohimbine is about 18 mg per day.

26. A method for supporting weight loss in a subject comprising providing to said subject the composition of claim 21.

27. The method of claim 26 wherein the composition further comprises yohimbine.
